(19) United States
(12) Patent Application Publication
(10) Pub. No.: US 2026/0224816 A1
(43) Pub. Date: Aug. 6, 2026

(54) SYRINGE WITH HANDLE

(71) Applicant: Paul Madan, University Heights, OH (US)

(72) Inventor: Paul Madan, University Heights, OH (US)

(21) Appl. No.: 19/046,560

(22) Filed: Feb. 6, 2025

(51) Int. Cl. A61M 5/315; A61M 5/00; B65D 83/76

(52) U.S. Cl. CPC A61M 5/31505; A61M 5/00; B65D 83/76; A61M 2005/31508; A61M 2209/08

(57) ABSTRACT

A syringe includes a barrel having a nozzle and an open end, and a plunger movably located within the barrel for dispensing a flowable material through the nozzle. A cover with a guide opening for the plunger can enclose the open end of the barrel. The plunger includes at least one and preferably a pair of tabs at a distal end of the plunger opposite the nozzle, with each tab having a catch on a side of the tab. A handle is attached to a distal end of the plunger, and has a cavity with a pair of shoulders positioned for engagement by the catches for retaining the handle on the plunger. A cap is inserted into an outer opening to the cavity, and includes a pair of projections for engaging a side of the tabs opposite the catches to prevent the catches from being removed from the shoulders.

### TECHNICAL FIELD

The present invention relates generally to syringes for dispensing flowable material.

### BACKGROUND

Syringes are commonly used for dispensing liquids and other flowable materials, such as in dispensing liquid medicines in the medical field, glues, adhesives, lubricants, sealants or paints in the construction and automotive fields, and in many applications around the home and business. Syringes typically include a barrel with a dispensing nozzle at one end, and a plunger which is forced forwardly against flowable material in the barrel to dispense the material out through the nozzle. With larger capacity barrels, a cover can be threaded onto the open end of the barrel opposite from the nozzle to facilitate loading the barrel with the material to be dispensed and to guide the shaft of the plunger within the barrel. The shaft of the plunger projects outwardly through a central opening in the cover, and a handle is provided at the opposite end of the plunger upon which pressure is applied to move the plunger forwardly within the barrel. The inner end of the plunger includes an enlarged head typically with a rubber sealing gasket. The gasket is closely received in the barrel and wipes against the inner surface of the barrel when the plunger moves to prevent the material leaking around the head. Lower cost syringes are often constructed primarily or wholly out of plastic, while higher cost syringes are often constructed out of multiple materials including plastic and metal.

When dispensing large quantities of a flowable material, or when the flowable material is less viscous, considerable pressure must be applied against the handle of the plunger in order to dispense the material through the nozzle. It can become exhausting and frustrating for a user to repeatably move the plunger within the syringe. Simmonds, U.S. Pat. No. 2,902,034, for example, shows a syringe with a flat, circular handle at the end of the plunger. The flat, circular handle can be uncomfortable to the user as the handle does not fit smoothly against the hand of the user. As a result, the user can become fatigued or sore during the dispensing process, and can even cock or tilt the plunger within the barrel of the syringe, which can lead to the uneven dispensing of material.

While it is known to provide a handle for a syringe with an ergonomic shape, such handles can be complicated and expensive to manufacture. And due to manufacturing, assembly and material limitations, particularly with the commonly-used two-part molding process, it can be difficult to provide an all-plastic integrated handle and plunger for larger syringes. As such, larger syringes typically have a multi-piece handle assembly, with a metal plunger shaft and a separate plastic or metal handle, where the handle is fixed by screw threads, roll pin, adhesive or press fit to the end of the shaft of the plunger. But again, as can be appreciated, this increases the over-all cost of the syringe when the plunger shaft and/or the handle are made from metal.

As such, it is believed there is a demand in the industry for a syringe that addresses at least some of the above issues and drawbacks.

### SUMMARY

A syringe is provided to dispense flowable materials. The syringe includes an ergonomic handle with a smooth, curved outer surface which comfortably fits the hand of the user such that the user can repeatably dispense the material without becoming fatigued or sore, and where the plunger and handle are manufactured from a relatively inexpensive material, such as plastic, and easily assembled into an integrated unit. The syringe is particularly useful for higher volume and/or lower viscous applications.

According to one aspect, the shaft of the plunger is formed from plastic or similar inexpensive material, and has a flexible latching device, which comprises, in one embodiment, at least one and preferably a pair of flexible tabs projecting axially outwardly from the end of the shaft. The tabs each include inwardly or outwardly-directed catches projecting from a side of the tab at the distal end of each tab.

The handle is also formed from a plastic or similar inexpensive material. The handle includes a body with an outer smooth, ergonomic end surface, and has an internal cavity with an upper opening, and a lower opening configured to receive the latching device of the plunger. The handle has a cooperating latching device, comprising, in one embodiment, at least one and preferably a pair of shoulders located along interior walls in the cavity such that when the plunger shaft is inserted through the lower opening and brought together with the handle, the tab(s) flex and then latch onto the shoulder(s).

In one embodiment where the catches on the end of the tabs face radially outwardly, the cavity can include radially-inward directed shoulders. In another embodiment where the catches on the ends of the tabs face radially inwardly, the cavity can have radially-outward directed shoulders. In either case when the handle is brought together with the plunger shaft, the tabs(s) latch onto the shoulder(s) and securely retain the handle to the plunger.

The handle can be easily formed using an injection molding process for example, using a two-part molding process.

A cap is provided that can be snapped onto the body of the handle to cover the upper opening, and provide a continuous, substantially seamless outer surface. To prevent the catches from backing off and disengaging from the shoulders during use, the cap includes at least one projection such as a rib, tab or flange located such that the projection is adjacent, and preferably bears against a side of a tab opposite from the catch when the cap is assembled with the handle. The tabs are effectively held between the projection of the cap, and the shoulders of the handle, when the cap is fixed to the handle.

The plunger and handle can be incorporated into a syringe having a barrel, and an enlarged plunger head with a resilient sealing gasket. The syringe can optionally include a cover at the open end of the barrel. The handle is easy and relatively low-cost to manufacture and assemble with the plunger, and the ergonomic configuration of the handle provides a comfortable experience to the user-even during repeated use in larger syringes dispensing larger quantities of flowable materials and/or where the materials are less viscous, and greater efforts are needed to dispense the material.

One additional feature, which can be used in addition or alternatively with the plunger and handle described above, is that the shaft of the plunger can have openings along its length, one of which can have a shape sized to receive the nozzle of the barrel. In such case, when the syringe is to be stored when not in use, the shaft can be removed from the barrel and located on the nozzle of the barrel. This has advantages, such as i) the components do not become separated and lost during storage; ii) the gasket does not dry and seize within the barrel; and iii) the gasket maintains a proper annular configuration without setting with a flat surface against the barrel, which could reduce its sealing capacity over time.

Additional features and advantages should become apparent to those skilled in the art upon reviewing the figures and following description.

### BRIEF DESCRIPTION OF THE DRAWINGS

Embodiments will now be described in further detail with reference to the accompanying drawings, in which:

FIG. 1 is a perspective view of an exemplary syringe constructed according to the principles of the present invention, illustrated with a plunger fully inserted into the barrel of the syringe and with a security strap;

FIG. 2 is a perspective view of the syringe of FIG. 1, illustrating the plunger in a retracted position;

FIG. 3 is an exploded view of the syringe of FIG. 1;

FIG. 4 is a cross-sectional side view of the handle assembly for the syringe of FIG. 1 attached to the end of the plunger;

FIG. 5 is side view of the end of the syringe of FIG. 1, showing the handle assembly in phantom;

FIG. 6 an elevated perspective view of the end of the syringe of FIG. 1, showing the handle assembly apart from the plunger;

FIG. 7 is an end view of the syringe of FIG. 1;

FIG. 8 is side view of the syringe of FIG. 1, shown in a stored position with the plunger; and

FIG. 9. is a top view of the syringe and plunger of FIG. 8.

### DETAILED DESCRIPTION

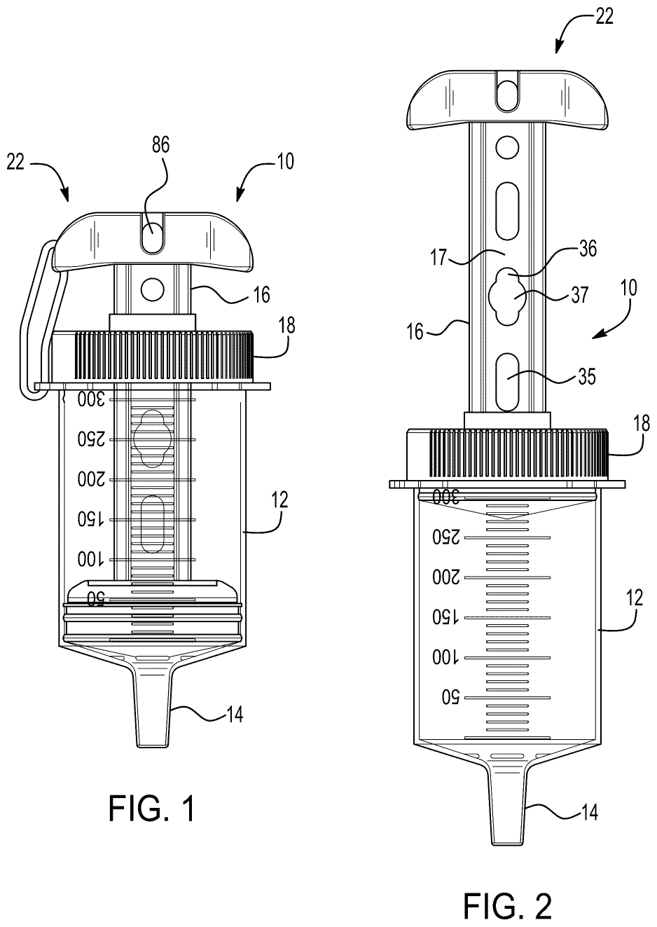

Referring to the drawings, and initially to FIGS. 1-3, an exemplary syringe is indicated generally at 10. The syringe includes an elongated, cylindrical barrel 12 with an elongated nozzle 14 at one end of the barrel, a plunger 16 with an elongated shaft 17, and optionally, a cover 18. A handle as at 22 is located on a distal end of the shaft 17 extending outwardly from the barrel, and at an end of the barrel opposite from the nozzle 14.

The syringe 10 is useful for a variety of applications to dispense a flowable material, such as to dispense liquid medicines in the medical field, oils and paints in the construction and automotive fields, and in many applications around the home and business in which it is desired to dispense a flowable material, such as a liquid. Such useful applications should be apparent to those skilled in the art.

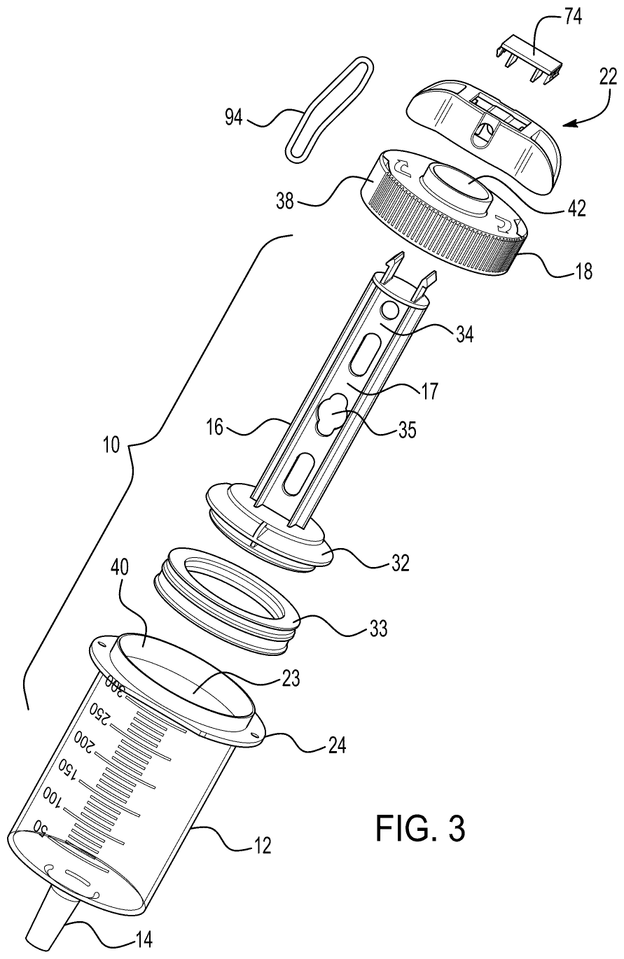

The barrel 12 of the syringe is typically formed from a plastic or other inexpensive lightweight material appropriate for the material to be dispensed. The barrel has a generally cylindrical configuration, with an open end 23 (FIG. 3) opposite from nozzle 14. The barrel can have a radially-outward projecting flange 24 near the open end with one or more flats 25 (FIG. 7) to facilitate holding the syringe during use and to prevent the syringe from rolling away when the syringe is put down on a surface. The barrel 12 is typically semi or fully transparent so that the user can visually inspect the amount of material in the barrel, and can have graduations or other markings to assist in determining the amount of material to be dispensed, as is known.

As shown in FIG. 3, the plunger includes an enlarged head 32 at the inner end of the shaft. An annular gasket 33 is sized to fit over the enlarged head of the plunger and is retained thereon in a known manner, such as by friction fit, adhesives, etc. The gasket 33 is outwardly sized so as to be closely received in the barrel 12, such that when the plunger is moved forwardly and backwardly within the barrel to dispense a material, the gasket sealingly wipes against the inner surface of the barrel and substantially prevents leakage of the material between the head of the plunger and the barrel. The body of the plunger and the gasket are also formed from an appropriate lightweight, inexpensive material appropriate for the material to be dispensed, for example, the body of the plunger can be formed from plastic while the gasket is formed from an elastomer or other resilient material.

The shaft 17 of the plunger can have a generally flat central body portion 34, which includes openings as at 35, to reduce the material necessary to form the plunger and to reduce the weight of the syringe. The openings 35 are generally evenly-spaced along the shaft 17, and can be elongated, circular, or in other geometries. One or more of the openings toward the center of the shaft 17, such as opening 36, can have an enlarged central portion 37. The central portion has a generally circular or keyhole configuration, the reason for which will be discussed below regarding the storage of the barrel and plunger.

Preferably the enlarged head 32 of the plunger is formed in one piece (unitarily) with the plunger shaft 17; although it could also be formed separately and later attached such as with adhesive, threads or by other appropriate technique.

The cover 18 for the syringe includes an annular outer wall 38 with internal screw threads or other geometry (not shown) which cooperate with threads or other geometry 40 (FIG. 3) on the outer surface of the barrel 12 near the open end 23, to enable the cover to be screwed onto and off of the barrel. The cover 18 includes a central opening 42, sized so as to closely receive and support the shaft 17 of the plunger 16. The shaft 17 and opening 42 can be partially or wholly circular to smoothly guide the plunger as the plunger moves forwardly and rearwardly within the barrel, or can have a non-circular geometry so as to also prevent rotation of the plunger during use. The cover 18 can likewise be formed of an appropriate lightweight, inexpensive material, such as plastic. The cover 18 is useful for larger capacity syringes where the material is loaded into the syringe through the open end of the barrel before the plunger in inserted; and it is noted that such a cover may not be necessary in all applications, in which case the plunger could be self-guided or guided by other structure within the barrel.

The barrel 12, plunger 16 and cover 18 are sized appropriately to accommodate the desired quantity of substance to be dispensed, as should be apparent to those skilled in the art.

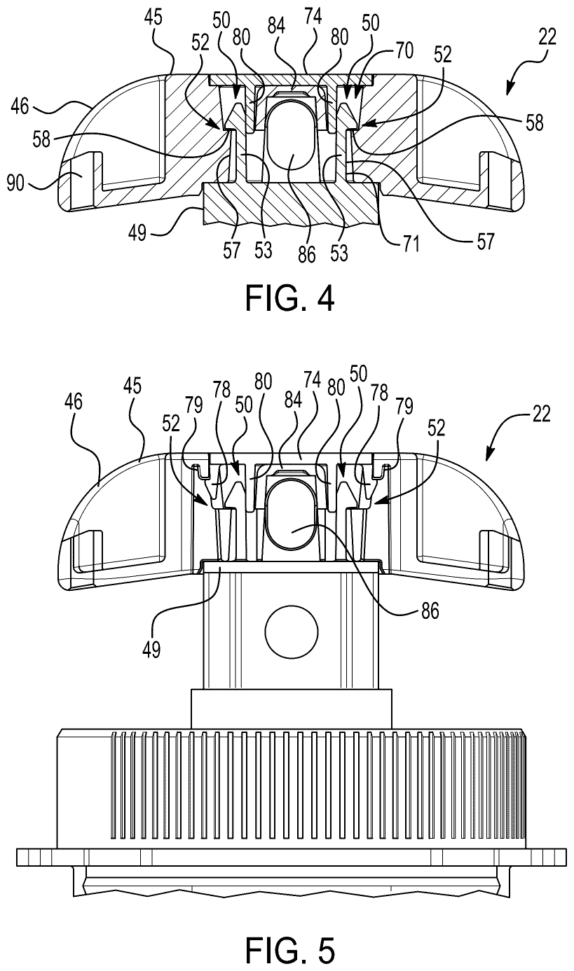

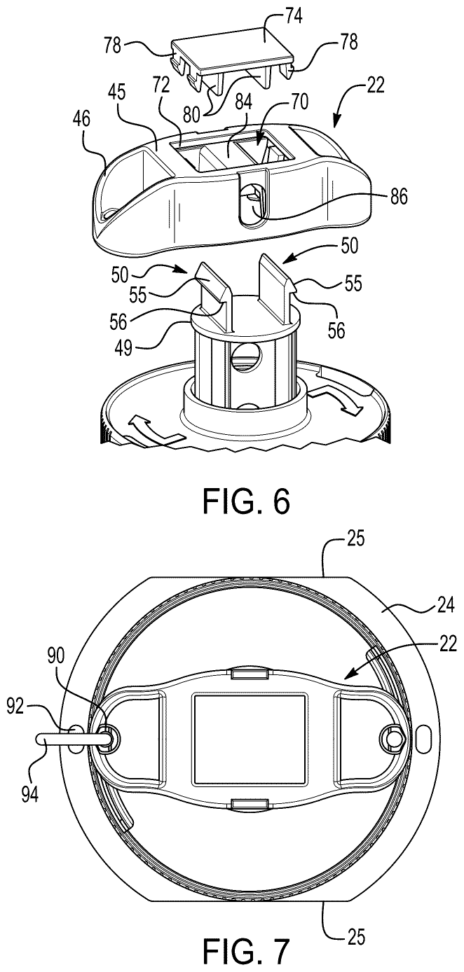

Referring now to FIGS. 4-6, the handle 22 for the syringe is illustrated with a body 45 having an ergonomic exterior configuration including a smoothly rounded or curved, outer end surface 46 configured to comfortably fit against the palm of a user's hand. The handle 22 is connected to the distal end 49 of the plunger shaft using one or more latching devices as at 50, which engage with one or more corresponding latching devices 52 on the handle. Specifically, according to one embodiment, the latching devices 50 on the plunger shaft comprise elongated flexible tabs 53, each of which projects axially outwardly from the end of the plunger, and each of which has a catch 55 (FIG. 6) which projects radially-outward from the distal free end of the tabs, along the outer side of the tab. The catch can be in the form of a wedge or other appropriate geometry as shown, having a ledge or shoulder 56 with a transverse, flat, inwardly-facing surface.

The latching devices 52 on the body of the handle can comprise a pair of spaced-apart, rigid interior walls 57 with opposing surfaces likewise having a ledge or shoulder 58 (FIG. 4) with a transverse, flat, outwardly-facing surface. The latching devices 50, 52 are dimensioned and located such that the catch 55 on each tab 53 can latch over and engage the shoulder 58 on a wall 57 of the body of the handle when the outer distal end 49 of the plunger is inserted into the body of the handle.

While a single latching device combination, such as a single tab-and-shoulder connection, may be appropriate for some applications, it is believed at least a pair of latching device combinations, such as the pair of tabs and shoulders as shown in FIGS. 4-6, will be appropriate to fix the plunger securely and stably to the handle in most applications. In such case, the tabs and shoulders are located such that the tabs are closely received between the interior walls 57 of the outer surface of the handle, with each wedge-shaped catch 55 riding up against an interior wall portion, flexing the tabs 53 radially inwardly until the catches 55 pass the shoulders 58 of the wall as the plunger is inserted into the handle and the tabs return to their normal orientation with the catches latched over the shoulders, with the result that the transverse, inwardly-facing surface of the shoulder of each catch 55 will be in opposed engagement against a transverse, outwardly-facing surface of a respective shoulder 58 on the wall of the body of the handle.

The tabs and shoulders are appropriately located on the end of the plunger shaft and in the handle, and can have appropriate dimensions and geometries, depending on the size of the plunger shaft and the handle and the anticipated pressures with the dispensing application.

Also, while the catches 55 are illustrated projecting from a radially outer side of the tab, and the interior walls 57 are illustrated with radially-inward facing shoulders, it should be appreciated that this could be reversed; that is, with the catches 55 provided on a radially inner side of the tabs, and the interior walls 57 located interiorly of the catches and having a radially-outward facing shoulder, with the same latching effect.

Still further, while the tabs are shown as comprising essentially straight, spaced-apart parallel walls projecting across a portion of the end of the plunger, and the interior walls are likewise shown in essentially straight, parallel, spaced-apart relation, the tab and shoulders could have other configurations, such as angled or curved. In an alternative embodiment, a single, flexible latching device having an annular configuration could be provided on the end of the plunger shaft, which would engage with a corresponding rigid annular latching device on the handle (either radially inward of the latching device of the plunger shaft, or radially outward depending on the geometry of the corresponding latching devices). In any event, the latching devices have cooperating geometry, such that when the latching device(s) are engaged, the handle is securely fixed to the end of the plunger shaft and preferably cannot be removed.

Referring to FIGS. 4 and 5, the interior walls 57 are formed in a cavity, indicated generally at 70, in the body 45 of the handle. The plunger 16 is inserted into an open lower end 71 of the cavity with the latching devices on the plunger shaft engaging the corresponding latching devices on the handle.

The cavity 70 also has an opening 72 (FIG. 6) on the outer, upper surface of the handle. To provide a smooth exterior surface on the handle, the handle can have a cap 74, which is sized so as to enclose and be completely and snugly received within the opening 72. The cap 74 can have spring tabs as at 78, appropriately sized and placed so that the tabs engage shoulders 79 (FIG. 5) along the interior walls 57 adjacent the opening 72 for a snap-fit. Other techniques could be used to secure the cap 74 to the handle, such as friction fit, or adhesive.

To prevent the tabs 53 from being released from the shoulders 58 during use, the cap can include one or more projections as at 80, extending inwardly from the inside surface of the cap and located so as to be closely received along and preferably bear against the inside surface of the tabs 53, opposite from the catches 55. The projections 80 can be in the form of tabs or ribs or other appropriate geometry, which prevent the tabs 53 of the latching device 50 from flexing radially inwardly during use and the catches releasing from the engaged connection with the shoulders 58 on the body of the handle. At least one projection 80 can be provided, and preferably a pair of projections are provided, with each projection located adjacent a respective tab 53 of the latching device 50.

The body of the handle can also include a U-shaped support beam 84, extending across the cavity 70 in the body. The support beam is dimensioned so as to closely support an inner surface of the projections 80 of the cap, and hence provide further support against the tabs 50 flexing and releasing from the shoulders 58.

In an alternate embodiment described above where the catches and shoulders could be reversed; that is, with the tabs 53 spaced radially outward from the interior walls, the projection(s) 80 from the cap can be located so as to be closely received along the outer surface of the tabs opposite from the catches 55. The projections would prevent the tabs from flexing outwardly in this case, and releasing from the shoulders.

The U-shaped support beam 84 also provides internal support for the body of the handle and allows a channel or opening 86 to be formed transversely across the handle. The opening 86 is sized and located to receive a hanger from a point-of-sale display case, so that the syringe can be hung on a display case for marketing and sale.

If desirable, the body of the handle can include further opening(s) 90 (FIGS. 4 and 7) at an appropriate location, such as toward an outer, upper end of the handle, and the flange 24 on the barrel can likewise include opening(s) 92 (FIGS. 3 and 7) at an appropriate location, and a plastic security strap 94 (FIGS. 1, 3 and 7) can be looped through the openings 90, 92 to prevent the syringe from being disassembled during marketing and display, until the strap is cut after purchase.

The body of the handle and the cap are likewise made from an inexpensive, lightweight material, such as plastic, and the body can be manufactured using known techniques, such as injection molding using a two-part molding process.

Referring now to FIGS. 3-6, during assembly of the syringe 10, the distal end 49 of the plunger 16 is inserted through the guide opening 42 in the cover 18, and the plunger is then inserted into the open lower end 71 of the cavity 70 in the handle. Once the catches 55 are firmly secured over the shoulders 58, the cap 74 can be snapped onto the outer opening 72 in the handle to complete the handle assembly. Material to be dispensed can be loaded into the open end 23 of the barrel, and the plunger 16 can then be inserted into the barrel and the cover 18 screwed onto the end of the barrel. Pushing the handle 22 of the plunger axially forwardly in the barrel dispenses the material through the nozzle 14 in a known manner.

In an appropriate application, the handle 22 can be pulled in an axially outward direction within the barrel, thus retracting the plunger within the barrel and creating a vacuum at the nozzle, which would allow material to be pulled into the barrel of the syringe through the nozzle for subsequent dispensing. This should also be well-known by those skilled in the art.

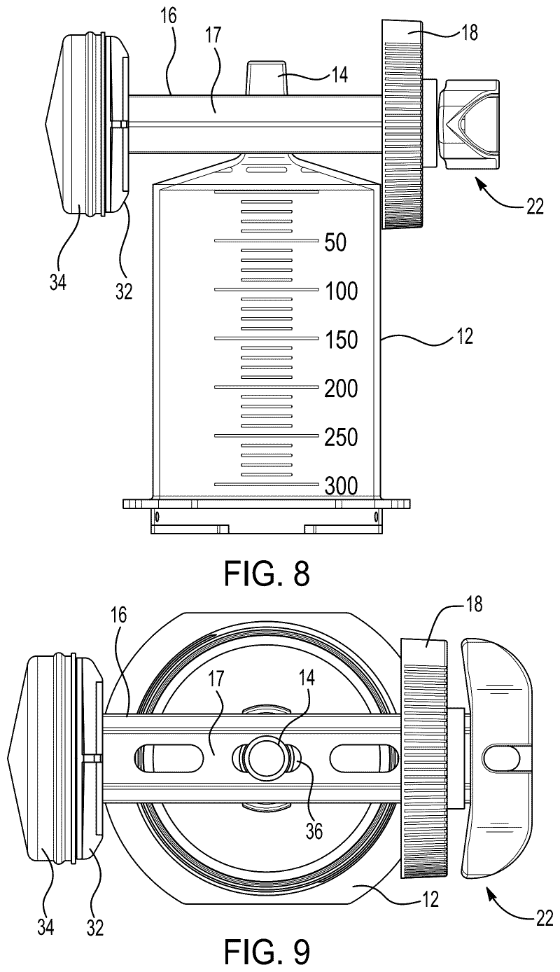

Referring now to FIGS. 8 and 9, one additional feature is illustrated which can be useful when storing the barrel and plunger of the syringe when not in use. As described previously, one or more of the openings along the shaft 17 of the plunger, for example opening 36, can have a shape which is sized to receive the nozzle of the barrel. Preferably opening 36 is at or near the midpoint of the shaft 17, or at least at or about the center of gravity of the plunger including cover 18 and handle 22, and has an enlarged, central circular or keyhole aperture 37. The central aperture 37 is sized to closely receive the nozzle 14 of the barrel 12 so that plunger fits snugly down along the nozzle, preferably at least half-way or more.

After the syringe has been used for a project, the plunger is removed from the barrel and the plunger and barrel are cleaned to remove paint or other residue. The barrel of the syringe can then be located in an upright, vertical manner on a shelf or other surface, and the plunger can be located on the nozzle of the barrel such that the nozzle is received through the aperture in the shaft to rest the plunger horizontally on the barrel. This prevents their separation and ease of finding for the next project. Also, as the gasket on the plunger is stored outside of the barrel, the gasket does not dry and seize within the barrel and maintains a proper annular configuration without setting with a flat surface against the barrel, which could reduce its sealing capacity over time.

Such storage feature can be used in conjunction or alternatively with the plunger and barrel described above.

As should be appreciated from the above, the handle assembly is relatively easy and low-cost to manufacture and assemble with the plunger, and the ergonomic configuration of the handle provides a comfortable experience to the user-even during repeated use in larger syringes dispensing larger quantities of flowable materials and/or where the materials are less viscous, and greater efforts are needed to dispense the material.

Although the principles, embodiments and operation of the syringe have been described in detail herein, this is not to be construed as being limited to the particular illustrative forms disclosed. They will thus become apparent to those skilled in the art that various modifications of the embodiments herein can be made without departing from the spirit or scope of the invention.

1. A syringe, comprising:
a barrel having a nozzle and an open end;
a plunger movably located within the barrel for dispensing a flowable material through the nozzle, the plunger having at least one flexible plunger latching device at a distal end of the plunger opposite from the nozzle, the at least one flexible plunger latching device having a catch projecting from a side of the at least one flexible plunger latching device;
and
a handle attached to the distal end of the plunger, the handle having at least one handle latching device, the catch of the at least one flexible plunger latching device engaging the at least one handle latching device for retaining the handle to the plunger, the handle further including a cap, the cap including a projection located adjacent the at least one flexible plunger latching device on another side of the at least one flexible plunger latching device opposite from the catch for preventing the catch from disengaging from the at least one handle latching device.

2. The syringe of claim 1, wherein the handle includes a body with a cavity having an opening on an outer surface of the handle opposite from the plunger, and the at least one handle latching device is located in the cavity.

3. The syringe of claim 2, wherein the cap encloses the opening in the body of the handle.

4. The syringe of claim 3, wherein the cap includes at least one projection, the projection located in adjacent relation to the at least one flexible plunger latching device on another side of the at least one flexible plunger latching device opposite from the catch for preventing the at least one flexible plunger latching device from disengaging from the at least one handle latching device.

5. The syringe of claim 1, wherein the handle has an external, rounded surface to provide a comfortable grip by a user.

6. The syringe of claim 1, wherein the at least one handle latching device is rigid.

7. The syringe of claim 1, wherein the at least one flexible plunger latching device comprises an elongated tab, and the at least one handle latching device comprises an interior wall with a shoulder.

8. The syringe of claim 1, wherein the at least one flexible plunger latching device comprises a pair of tabs at the distal end of the plunger with each tab having a catch projecting from a side of each respective tab, and the at least one handle latching device comprises a pair of interior walls, each with a shoulder, the shoulders being located such that each of the catches engages a respective shoulder.

9. The syringe of claim 8, wherein the cap includes a pair of projections, with each projection located in bearing relation against a respective catch on the other side of the respective tab opposite from the catch.

10. The syringe of claim 1, further comprising a cover attached to the open end of the barrel, the cover having a guide opening receiving the plunger and in which the plunger is guided during movement of the plunger in the barrel.

11. The syringe of claim 1, wherein the handle has a transversely-extending hole through which a display hanger can pass for hanging the syringe from a point-of-sale display.

12. A syringe, comprising:
a barrel having a nozzle and an open end;
a plunger movably located within the barrel for dispensing a flowable material through the nozzle, the plunger having a latching device at a distal end of the plunger opposite from the nozzle; and
a handle attached to the distal end of the plunger, the handle also having a latching device, in engagement with the latching device of the plunger for retaining the handle to the plunger against separation, one of the latching devices of the plunger and of the handle being flexible, and the handle further including a cap, the cap including a projection located adjacent the one flexible latching device, on a side of the one flexible latching device opposite from the other of the latching devices.

13. The syringe of claim 12, wherein the one flexible latching device includes a catch on another side of the one flexible latching device opposite from the projection.

14. The syringe of claim 13, wherein the other of the latching devices comprises an interior wall with a shoulder, the catch on the one flexible plunger latching device engaging the shoulder of the other latching device.

15. The syringe of claim 14, wherein the handle includes a body with a cavity having an opening on an outer surface of the handle opposite from the plunger, and the other of the latching devices is located in the cavity.

16. The syringe of claim 15, wherein the cap encloses the opening in the body of the handle.

17. A syringe, comprising:
a barrel having a nozzle and an open end;
a plunger movably located within the barrel for dispensing a flowable material through the nozzle, the plunger having a shaft with one or more elongated openings along a length of the shaft, at least one elongated opening of the one or more elongated openings sized to closely receive the nozzle of the barrel and having an enlarged central circular or keyhole aperture; and
a handle attached to a distal end of the plunger.

18. A method for storing the plunger on the barrel of a syringe, the syringe including a barrel having a nozzle and an open end; a plunger movably located within the barrel for dispensing a flowable material through the nozzle, the plunger having a shaft with one of more openings along the length of the shaft, at least one opening of the one or more openings sized to closely receive the nozzle of the barrel; and a handle attached to a distal end of the plunger, the method comprising the steps of:
i. removing the plunger from the barrel;
ii. supporting the barrel in an upright, vertical manner; and
iii. inserting the nozzle of the barrel into the at least one opening to rest the plunger horizontally on the barrel.
